(19) United States
(12) Patent Application Publication
(10) Pub. No.: US 2016/0199172 A1
(43) Pub. Date: Jul. 14, 2016

(54) TENDON REPAIR SYSTEMS, DEVICES, AND KITS

(71) Applicant: CAPERE SURGICAL SYSTEMS PTE LTD., Nanyang Drive, Singapore (SG)

(72) Inventors: Beng Hai Lim, Singapore (SG); Siaw Meng Chou, Singapore (SG)

(73) Assignee: CAPERE SURGICAL SYSTEMS PTE LTD., Singapore (SG)

(21) Appl. No.: 14/912,640

(22) PCT Filed: Aug. 18, 2014
(86) PCT No.: PCT/SG2014/000388

§ 371 Date: Feb. 18, 2016

Related U.S. Application Data
(60) Provisional application No. 61/867,581, filed on Aug. 19, 2013.

(51) Int. Cl. A61F 2/08

(52) U.S. Cl. CPC A61F 2/0811; A61F 2/08; A61F 2/0805; A61F 2220/0016

(57) ABSTRACT

A tendon overlay device includes an arcuate main body having an outer surface and an inner surface, each of the outer surface and the inner surface extending between a first terminal edge of the main body and a second terminal edge of the main body; a first plurality of teeth disposed along the first terminal edge of the main body; and a second plurality of teeth disposed along the second terminal edge of the main body. Each tooth within the first plurality of teeth projects downward away from the first terminal edge of the main body and is biased inward toward the second plurality of teeth. Each tooth within the second plurality of teeth projects downward away from the second terminal edge of the main body and is biased inward toward the first plurality of teeth. The inner surface of the main body excludes spikes and serrations that can penetrate into an outer surface of a tendon on which the inner surface of the main body is disposable.

### TECHNICAL FIELD

The present disclosure relates generally to implantable devices for tendon repair, such as hand flexor or extensor tendon repair. Aspects of the present disclosure are directed to tendon overlay devices that are implantable on each side of a tendon repair site. Each tendon overlay device includes (a) a main body providing a substantially semi-cylindrical profile configured for partially encircling a tendon; (b) teeth extending away from terminal edges of the main body and directed inwardly toward a midline of the main body, where such teeth are configured for penetrating into portions of the tendon to a predetermined depth; and (c) a plurality of openings through which sutures can be passed or routed.

### BACKGROUND

Multiple approaches exist for repairing tendon lacerations. For instance, two segments of a tendon can be joined together across a laceration solely by way of sutures, which are routed or organized in accordance with a suture pattern that is expected to provide high tensile strength and mechanically reliability. Several suture patterns have been developed for tendon repair, including the well known modified Kessler technique. Unfortunately, sutures alone often fail to provide adequate strength and reliability needed for rehabilitation activities involving the injured tendon. As a result, implantable tendon repair devices have been developed.

One type of implantable tendon repair device, which is particularly directed to hand flexor tendon repair, provides anchor structures that are internally implanted within each tendon segment, on opposite sides of a laceration. A pair of implanted intra-tendon anchors are joined together by a multi-filament stainless steel suture. While such an anchored approach provides high tensile strength and mechanical reliability, the implantation of the anchors is not a simple procedure. Furthermore, in the event that the tendon becomes infected around or proximate to one or both intra-tendon anchors, removal of the anchors without imparting significant or severe damage to the tendon is generally not possible, and cutting of the tendon to remove the anchors is typically required, thereby negating the original repair.

Another type of implantable tendon repair device is disclosed in U.S. Pat. No. 5,916,224, which describes a tendon repair clip that is designed to be implanted such that it wraps around or surrounds the majority of a tendon's outer circumference. The tendon repair clip includes two serrated sections that are integrally joined to a hinged or deformable connecting section disposed therebetween, such that the serrated sections are laterally adjacent to the connecting section. The connecting section has a predetermined curvature. Each serrated section includes an approximately flat region that includes a plurality of spikes that project downwardly from the serrated section's underside. Additionally, each serrated section is joined to a substantially straight leg member that projects downward, and which carries a plurality of teeth at a distal end. Furthermore, each serrated section includes a suture opening.

A tendon laceration is repaired by way of implanting two tendon repair clips, where one tendon repair clip is implanted on a tendon segment on one side of the laceration, and the other tendon repair clip is implanted on the tendon segment on the other side of the laceration. With respect to the dorsal implantation of a given tendon repair clip on a tendon segment, the clip's connecting section has a smooth underside, and is intended to be positioned above the dorsal aspect of the tendon segment, along which the tendon's vascular supply resides. Once the connecting section is in position, pressure can be applied to the clip such that the spikes on the undersides of the serrated sections penetrate into the tendon, substantially or immediately adjacent to the tendon's dorsal aspect, on opposite sides of the tendon's central dorsal vascular supply. As part of the implantation procedure, each tendon repair clip is mechanically deformed or folded axially about its connecting section, such that the leg members wrap around lateral and volar portions of the tendon segment on which the repair clip is implanted. Following such axial folding, the teeth at the end of each leg member are crimped in an alternating sequence, such that each tooth penetrates into a volar portion of the tendon. Finally, after all teeth have been alternately crimped on each tendon repair clip, and each tooth penetrates into its tendon segment, sutures can be routed through the tendon segments and the suture openings in the serrated sections of each tendon repair clip, such that the tendon segments can be drawn together.

Unfortunately, mispositioning of a tendon repair clip on a tendon segment during implantation can result in a serrated section's spikes penetrating into the tendon's vascular supply, thereby damaging the tendon and reducing the likelihood of successful healing. Additionally, the multi-step implantation procedure is undesirably time consuming and complex. Furthermore, once a tendon repair clip is implanted, removal of the tendon repair clip from a tendon segment, if required, is undesirably difficult.

A need exists for an implantable tendon repair device that overcomes the deficiencies indicated above.

### SUMMARY

In accordance with an aspect of the present disclosure, a tendon overlay device includes: an arcuate main body having an outer surface and an inner surface, each of the outer surface and the inner surface extending between a first terminal edge of the main body and a second terminal edge of the main body; a first plurality of teeth disposed along the first terminal edge of the main body; and a second plurality of teeth disposed along the second terminal edge of the main body. Each tooth within the first plurality of teeth projects downward away from the first terminal edge of the main body and is biased inward toward the second plurality of teeth. Each tooth within the second plurality of teeth projects downward away from the second terminal edge of the main body and is biased inward toward the first plurality of teeth. In various embodiments, the inner surface of the main body excludes spikes and serrations that can penetrate into an outer surface of a tendon on which the inner surface of the main body is disposable.

In some embodiments, a central axis of the main body is definable which bisects the main body into symmetric arcuate halves, and wherein each tooth within each plurality of teeth is biased inward toward the central axis.

Several embodiments of the device have each symmetric arcuate half shaped in the form of a single arc.

Some embodiments of the device also have each tooth within each plurality of teeth biased inward toward the central axis, for instance, in accordance with a common angle.

In multiple embodiments, each of the outer surface of the main body and the inner surface of the main body form an arcuate segment of an ellipse or circle. In some embodiments, the ellipse or circle of the device has a predetermined dimension or diameter that is expected to substantially match a counterpart or corresponding dimension or diameter of a tendon of a particular size, such that the inner surface of the main body can matingly engage with a portion of a circumference of the tendon.

According to some embodiments, the main body has a semi-cylindrical shape along a main body length L, wherein the semi-cylindrical shape is defined by a constant radius of curvature between the first terminal edge and the second terminal edge, and wherein each symmetric half of the main body exhibits a shape corresponding to a quarter cylinder. The main body can have a thickness defined by a distance between the inner surface of the main body and the outer surface of the main body, wherein the thickness is between approximately 0.20 mm and 0.30 mm depending upon embodiment details.

In certain embodiments, the disclosed device has a ratio of a diameter corresponding to the arcuate main body and a distance to which each tooth within the first plurality of teeth and each tooth within the second plurality of teeth is configured to penetrate into a tendon is approximately 1.0 mm-1.3 mm.

In several embodiments, the main body of the device carries, includes, or is manufactured from a metal.

According to multiple embodiments, a device in accordance with the present disclosure further includes a first plurality of suture openings formed in the main body and disposed laterally away from the central axis of the main body proximate to the first terminal edge of the main body, above the first set of teeth; and a second plurality of suture openings formed in the main body and disposed laterally away from the central axis of the main body proximate to the second terminal edge of the main body, above the second set of teeth. Each of the first and second pluralities of suture openings include openings having at least one of different shapes and different cross sectional areas in some embodiments.

One aspect of the present disclosure involves a tendon repair structure including: a first tendon overlay device disposed on a first segment of a tendon corresponding to a first side of a laceration in the tendon; a second tendon overlay device disposed on a second segment of the tendon corresponding to a second side of the laceration in the tendon; and at least one set of sutures coupling the first tendon overlay device to the second tendon overlay device; Further, each of the first and second tendon overlay devices includes: an arcuate main body having an outer surface and an inner surface, each of the outer surface and the inner surface extending between a first terminal edge of the main body and a second terminal edge of the main body; a first plurality of teeth disposed along the first terminal edge of the main body; and a second plurality of teeth disposed along the second terminal edge of the main body, wherein each tooth within the first plurality of teeth projects downward away from the first terminal edge of the main body and is biased inward toward the second plurality of teeth, and wherein each tooth within the second plurality of teeth projects downward away from the second terminal edge of the main body and is biased inward toward the first plurality of teeth. The inner surface of the main body excludes spikes and serrations that can penetrate into an outer surface of a tendon on which the inner surface of the main body is disposable.

In multiple embodiments of the tendon repair structure, each of the first and second tendon overlay devices further includes one or more suture openings, e.g., a plurality of suture openings, through which the at least one suture can be routed such that a force applied to the at least one suture draws the first and second tendon overlay devices toward each other and brings the first segment of the tendon into intimate contact with the second segment of the tendon.

Pursuant to another aspect the present disclosure, a surgical kit for tendon repair includes at least one pair of tendon overlay devices. Each of the tendon overlay devices includes an arcuate main body having an outer surface and an inner surface, each of the outer surface and the inner surface extending between a first terminal edge of the main body and a second terminal edge of the main body; a first plurality of teeth disposed along the first terminal edge of the main body; and a second plurality of teeth disposed along the second terminal edge of the main body, wherein each tooth within the first plurality of teeth projects downward away from the first terminal edge of the main body and is biased inward toward the second plurality of teeth, and wherein each tooth within the second plurality of teeth projects downward away from the second terminal edge of the main body and is biased inward toward the first plurality of teeth. Each pair of tendon overlay devices within the at least one pair of tendon overlay devices corresponds to an ellipse or circle of a predetermined diameter that is expected to correspond or approximately correspond to the diameter of a mammalian tendon. The inner surface of the main body excludes spikes and serrations that can penetrate into an outer surface of a tendon when the tendon overlay device is applied to portions of a tendon.

In a number of embodiments, the ellipse or circle of a predetermined diameter of each tendon overlay device of the surgical kit corresponds to or is an ellipse circle that is expected to approximately correspond to a dimension or diameter of a human hand tendon within one of hand flexor tendon zone V, hand flexor tendon zones II to IV, and hand extensor tendon zones II to V.

The surgical kit for tendon repair, according to some embodiments, further includes at least one tendon sizing device having an arcuate measurement segment corresponding to an ellipse or circle of a predetermined dimension or diameter that is expected to approximately correspond to a dimension or diameter of a mammalian tendon. Accordingly, the arcuate measurement segment of the at least one tendon sizing device corresponds to an ellipse or circle that is expected to approximately correspond to a dimension or diameter of a human hand tendon within one of hand flexor tendon zone V, hand flexor tendon zones II to IV, and hand extensor tendon zones II to V.

Some embodiments of the disclosed surgical kit further include a tendon overlay device crimping tool configured for (a) holding a tendon overlay device in position around a portion of a circumference of a tendon, and (b) applying a force to an outer surface of the tendon overlay device to thereby simultaneously drive the first plurality of teeth and the second plurality of teeth into lateral portions of the tendon.

In several embodiments, the tendon overlay device crimping tool includes: a set of handles graspable by a user; a set of arm members coupled to the set of graspable handles; and a first closure arm and a second closure arm coupled to the set of arm members, wherein the first and second closure arms are configured for holding the tendon overlay device in position on the tendon and applying the force to the outer surface of the tendon overlay device in response to user interaction with the set of handles. Furthermore, in a number of embodiments the first and second closure arms are inwardly displaceable toward each other across a maximum crimping distance through which the closure arms can be displaced toward each other to laterally deform the tendon overlay device and simultaneously drive the first set of teeth and the second set of teeth into lateral portions of the tendon.

### BRIEF DESCRIPTION OF THE DRAWINGS

FIGS. 1A and 1B are schematic illustrations of a pair of tendon overlay devices implanted on a first tendon segment and a second tendon segment, respectively, on opposite sides of a tendon laceration.

FIGS. 2A-2C are schematic illustrations of representative tendon overlay devices in accordance with embodiments of the present disclosure.

FIG. 3 is a schematic illustration of a representative tendon sizing device in accordance with an embodiment of the present disclosure.

FIG. 4 is a schematic illustration of a representative tendon overlay device crimping tool in accordance with an embodiment of the present disclosure.

### DETAILED DESCRIPTION

In the present disclosure, depiction of a given element or consideration or use of a particular element number in a particular FIG. or a reference thereto in corresponding descriptive material can encompass the same, an equivalent, or an analogous element or element number identified in another FIG. or descriptive material associated therewith. The use of “/” in a FIG. or associated text is understood to mean “and/or” unless otherwise indicated. The recitation of a particular numerical value or value range herein is understood to include or be a recitation of an approximate numerical value or value range (e.g., within +/−10%).

As used herein, the term “set” corresponds to or is defined as a non-empty finite organization of elements that mathematically exhibits a cardinality of at least 1 (i.e., a set as defined herein can correspond to a unit, singlet, or single element set, or a multiple element set), in accordance with known mathematical definitions (for instance, in a manner corresponding to that described in An Introduction to Mathematical Reasoning: Numbers, Sets, and Functions, “Chapter 11: Properties of Finite Sets” (e.g., as indicated on p. 140), by Peter J. Eccles, Cambridge University Press (1998)). In general, an element of a set can include or be a system, an apparatus, a device, a structure, an object, a physical parameter, or a value depending upon the type of set under consideration.

Embodiments of the present disclosure are directed to systems, apparatuses, devices, and kits for tendon repair. Multiple embodiments are well suited for the repair of hand flexor or hand extensor tendons, for instance, hand tendon repair in flexor tendon zone V, as well as flexor tendon zones II to IV; and extensor tendon zones II to V.

Embodiments in accordance with the present disclosure include tendon overlay/repair devices, which are pairwise implantable relative to first and second sides of a tendon repair site corresponding to a laceration, gap, break, or discontinuity in a tendon under consideration. In various embodiments, each tendon overlay device is configured for covering or surrounding approximately one-half of a tendon's periphery or outer circumference. Each tendon overlay device includes a plurality of teeth that extend away from terminal edges or ends of the tendon overlay device at lateral sides of the tendon overlay device. The teeth are directed downward and inward with respect to a bisecting midline of the tendon overlay device, which is defined midway between the tendon overlay device's terminal edges. The teeth can penetrate into lateral portions of the tendon that are away from the tendon's dorsal aspect by way of a single step crimping procedure that secures or anchors the tendon overlay device to the tendon. Suture openings or holes provided in each tendon overlay device facilitate the routing of one or more sutures over and/or under the tendon overlay device. Consequently, a suturing procedure can draw or pull together a pair of tendon overlay devices secured to the tendon on opposite sides of the tendon repair site, thereby drawing separate segments or portions of the tendon into intimate contact at the laceration, while simultaneously distributing mechanical forces applied by the sutures across portions the tendon overlay devices. The anchoring of each tendon overlay device in the tendon by way of the teeth, in combination with suturing through/between the openings in each tendon overlay device, results in a high or very high tensile strength tendon repair structure that overcomes the drawbacks of prior tendon repair devices and techniques.

Aspects of Representative Tendon Overlay Devices

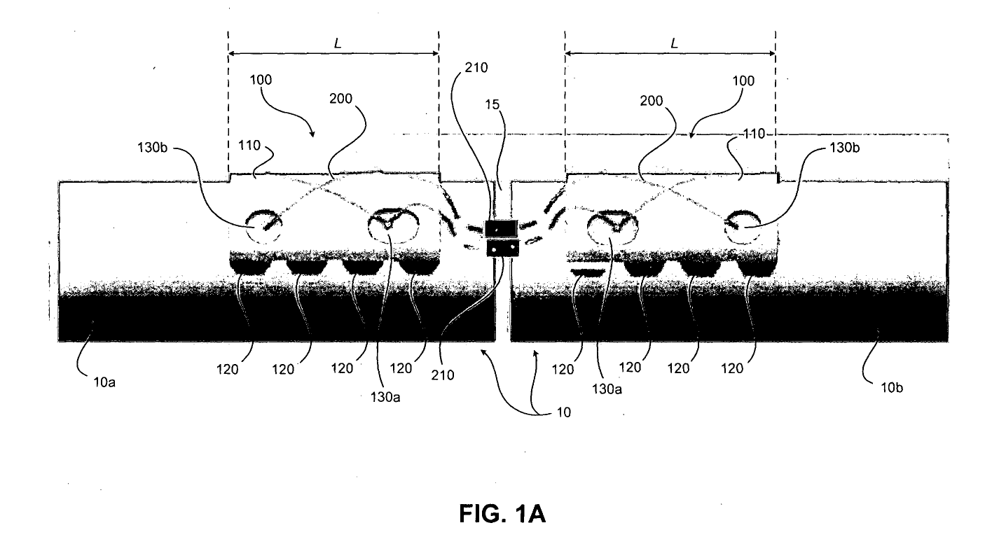

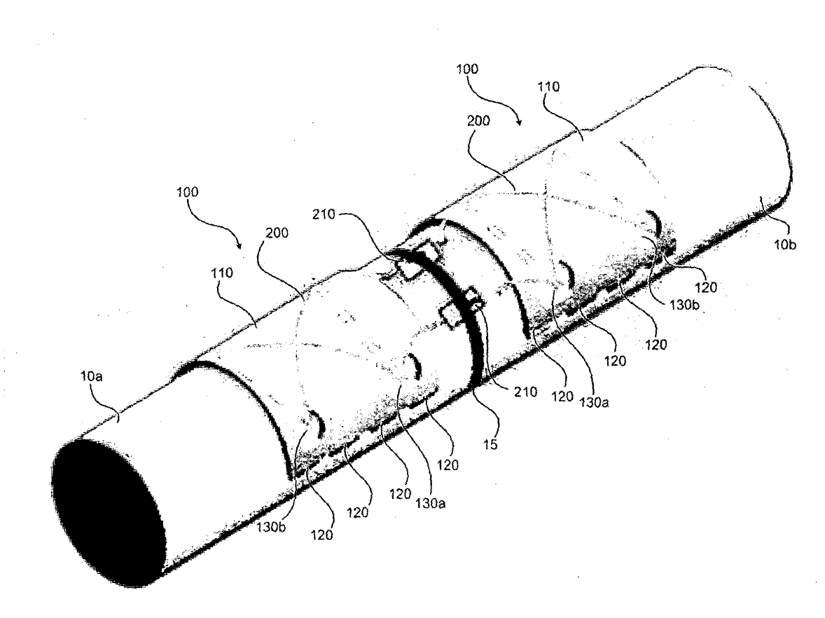

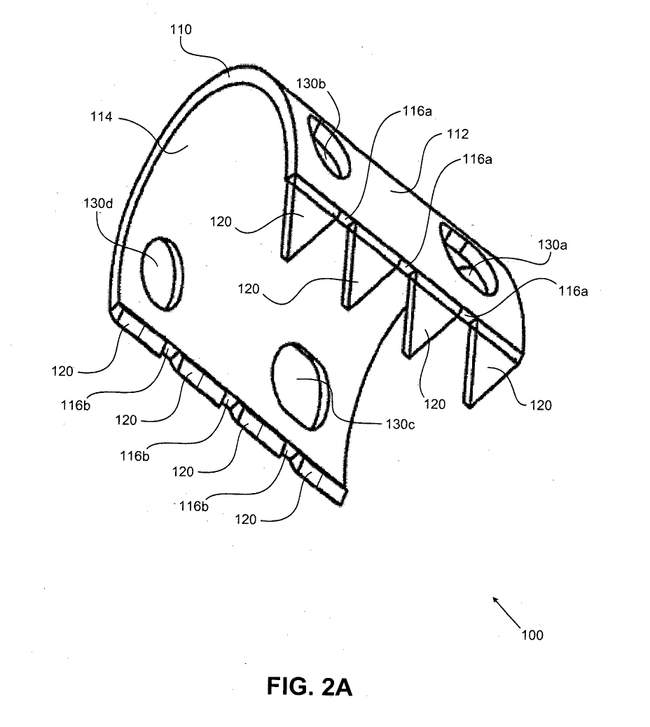

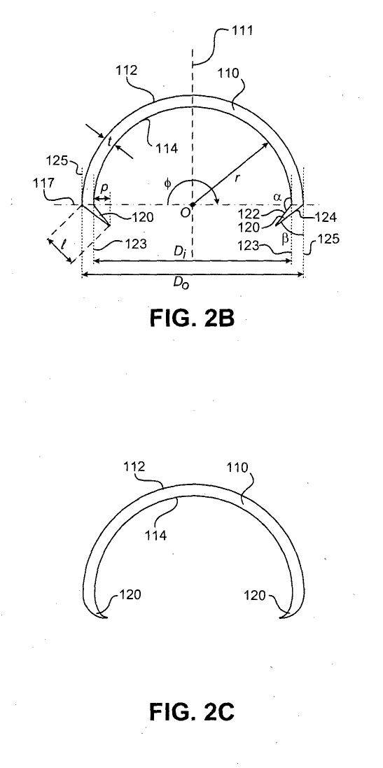

FIGS. 1A and 1B are schematic illustrations of a pair of tendon overlay/repair devices 100a,b implanted on a first segment/portion 10a and a second segment/ portion 10b of a tendon 10, respectively, on opposite sides of a laceration, break, discontinuity, or gap 15 in the tendon 10. FIGS. 2A-2C are schematic illustrations of representative tendon overlay devices 100 in accordance with embodiments of the present disclosure. Each tendon overlay device 100a,b includes a main body 110 having a length L and a substantially or essentially semi-cylindrical overall shape or profile along its length L. The main body 110 is configured (e.g., shaped and dimensioned) for surrounding or partially encircling approximately or essentially one-half of the tendon's exterior or circumference along the length L of the main body 110.

As indicated in FIG. 2A, the main body 110 includes an outer surface or upper side 112, and an inner surface or underside 114, each of which extends between a first terminal edge or end 116a and a second terminal edge or end 116b of the main body 110 to provide the tendon overlay device 100 with its substantially or essentially semi-cylindrical shape. As illustrated in FIG. 2B, a distance between the main body's outer surface 112 and its inner surface 114 defines a thickness t of the main body 110. Additionally, a midline or central axis 111 of the main body 110 can be defined, which bisects the main body 110 into two symmetric halves (e.g., each of which substantially or essentially exhibits a quarter cylinder shape). The first and second terminal edges 116a,b are thus disposed lateral to the main body's midline 111. In various embodiments, the first and second terminal edges 116a,b of the main body 110 can be defined to reside in a common plane 117 that is perpendicular to the main body's central axis 111, and which intersects the central axis 111 at a main body geometric center or origin O. Thus, in such embodiments an angular span Φ between the first terminal edge 116a of the main body 110 and the second terminal edge 116b of the main body 110 is approximately or essentially 180°. An inner radius having length r extending away from the origin O to the inner surface 114 of the main body 110 can also be defined. An inner diameter Dᵢ corresponding to the main body 110 is thus twice its inner radius r. In an analogous manner, an outer radius (not shown) having length (r+t) can be defined, and an outer diameter Dₒ corresponding to the main body 110 is twice its outer radius (or equivalently, twice its inner radius r plus twice its thickness t). In view of the foregoing, in multiple embodiments, the main body 110 exhibits a substantially or essentially constant radius of curvature.

When the main body 110 partially surrounds portions of a tendon 10, the main body's inner surface 114 contacts portions of the tendon's the exterior or outer surface. In various embodiments, the main body's inner surface 114 substantially, essentially, or entirely excludes or omits spikes, serrations, or protrusions that can penetrate into and potentially damage dorsal regions or portions the tendon 10. Thus, in various embodiments, while the main body's inner surface 114 can exhibit a certain degree of surface roughness, the inner surface 114 is sufficiently smooth between the main body's first and second terminal edges 116a,b that any significant penetration of portions of the main body's inner surface 114 into the tendon 110 is avoided.

Instead of spikes, serrations, or protrusions extending away from portions of the inner surface 114 between the main body's first and second terminal edges 116a,b, embodiments in accordance with the present disclosure carry a plurality of teeth 120 that extend downwardly away from each of the main body's first and second terminal edges 116a,b. The teeth 120 are angled or directed inward toward the main body's central axis 111. More particularly, in multiple embodiments, (a) an inner surface 122 of a given tooth 120 can be inwardly disposed approximately at an angle α relative to an inner tangent line 123 defined with respect to the main body's inner surface 110, where such inner tangent line 123 is perpendicular to the common plane 117 in which the main body's first and second terminal edges 116 reside; and (b) an outer surface 124 of the tooth 124 can be inwardly disposed approximately at an angle β relative to an outer tangent line 125 defined with respect to to the main body's outer surface 112, where such outer tangent line 125 is also perpendicular to this common plane 117 (and hence the outer tangent line 125 is parallel to the inner tangent line 123). A tooth 120 can have a length i along its outer surface, and the tooth 120 can project an inward distance p away from the main body's inner surface 114 at the main body's terminal ends 116a,b.

The teeth 120 are tapered to form sharp points (e.g., pointed ends) that facilitate penetration of the teeth 120 into the tendon 10 in response to the application of mechanical pressure or force to the outer surface 112 of the main body 110. Such pressure or force can be applied to the main body 110 during a single step crimping procedure directed to a given tendon overlay device 100, during which all teeth 120 on the main body 110 substantially or essentially simultaneously penetrate into the tendon 10 and anchor the main body 110 thereto, as further detailed below. In various embodiments, the teeth 120 are V-shaped; however, in other embodiments, the teeth 120 can exhibit other or additional shapes, for instance, in a manner depicted in FIG. 2C. Depending upon embodiment details, each tooth 120 can have a substantially or essentially identical shape, length i, and projection distance p away from the main body's inner surface 114; or one or more teeth 120 can have a shape, a length i, and/or a projection distance p that differs from other teeth 120. A tooth's projection distance p corresponds to the distance to which the tooth 120 laterally penetrates into the tendon 10.

As indicated in FIGS. 1A, 1B, and 2A, the main body 110 includes a plurality of openings or holes 130a-d therein, which are shaped and dimensioned to facilitate the passage or routing of tendon sutures 200 therethrough/therebetween. Such suture openings 130a-d are disposed toward or on lateral sides of the main body 110, above the teeth 120. In a number of embodiments, the main body 110 includes suture openings 130 of different sizes, areas, or shapes. For instance, suture openings 130a,c that are intended to be disposed proximal to the tendon laceration 15 can provide a larger cross sectional area, than suture openings 130b,d that are intended to be disposed distal to the tendon laceration 15; and/or a different shape (e.g., approximately elliptical) than distal suture openings 130b,c (e.g., approximately circular). Larger area/differently shaped proximal suture openings 130a,c can facilitate the formation or passage of multiple sutures 200 near the tendon laceration 15, in accordance with an intended or desired suture pattern that is expected to increase or enhance the tensile strength of the overall tendon repair structure, which includes a pair of tendon overlay devices 100a,b and sutures 200 routed through and between each tendon overlay device 100a,b. While a particular representative suture pattern is shown in FIGS. 1A and 1B, individuals having ordinary skill in the relevant art will understand that other types of suture patterns can be employed with tendon overlay devices 100a,b disposed on opposite sides of a tendon laceration 15 in accordance with embodiments of the present disclosure.

In representative implementations, tendon overlay devices 100 are manufactured (e.g., by way of a conventional stamping process) from at least one biocompatible metal, such as stainless steel (e.g., type 316 stainless steel), titanium alloy, magnesium alloy or cobalt-Chromium alloy (e.g., L605 or MP35N). The thickness of a tendon overlay device 100 can be, for instance, between approximately 0.20-0.30 mm (e.g., about 0.25 mm). In certain embodiments, tendon overlay devices 100 can include a biocompatible coating. In a representative implementation, the teeth 120 can have a length i of approximately 1.0-1.5 mm (e.g., 1.0-1.3 mm, 1.2 mm-1.4 mm, or 1.3 mm); and angles α and β can respectively be approximately 30° (e.g., 22.5°-37.5°, or 25°-35° and 45° (e.g., 40°-50°.

Because tendons 10 exhibit different sizes or diameters, different sets of tendon overlay devices 100 can be manufactured to have different inner diameters Dᵢ to facilitate size matching of the inner diameter Dᵢ of a given tendon overlay device 100 to the diameter of a tendon 10 under consideration. In representative implementations, different tendon overlay devices 100 can be manufactured to have inner diameters of approximately 5 mm, 6 mm, and 7 mm, to match tendons 10 having outer diameters of approximately 5 mm, 6, mm, or 7 mm, respectively. Tendon overlay devices 100 of a given inner diameter Dᵢ can form a portion of a tendon repair kit.

Aspects of Representative Implantation Procedures

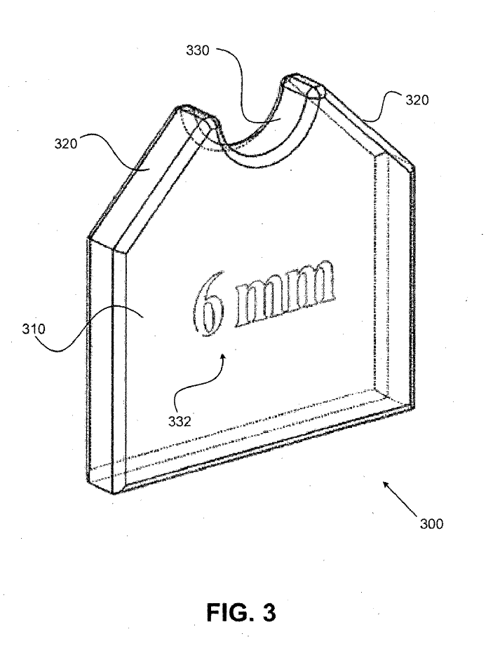

In an embodiment, tendon size or diameter measurement precedes the implantation of tendon overlay devices 100a,b on each side of a tendon laceration 15, such that appropriately sized tendon overlay devices 100 can be selected. Such measurement can occur by way of a tendon sizing device 300, a representative embodiment of which is shown in FIG. 3. The tendon sizing device 300 can include a graspable handle member 310, and tapered segments 320a,b projecting away from the handle member 310 toward and to a reference measurement arc or groove 330 disposed at a distal end of the tendon sizing device 300. The reference arc 300 is formed such that it forms a portion of a circumference corresponding to a known or predetermined diameter, such as 5 mm, 6 mm, or 7 mm. Reference diameter indicia 332 can be provided on an exterior surface of the handle member 310. A set of tendon sizing devices 300 can form a portion of a tendon repair kit in accordance with an embodiment of the present disclosure.

Tendon sizing occurs by way of positioning one or more tendon sizing devices 300 upon an exposed periphery of tendon 10 under consideration, and visually determining which tendon sizing device 300 provided a measurement arc or groove 330 that most closely or most uniformly matches or fits the tendon's periphery, in a manner readily understood by one of ordinary skill in the relevant art. Based upon which tendon sizing device 300 provided such a closest match or best fit with the tendon's periphery, a surgeon can select a pair of appropriately sized tendon overlay devices 100a,b that can individually reside on opposite sides of a laceration 15 in the tendon under consideration.

Following the selection of a pair of appropriately sized tendon overlay devices 100a,b, each tendon overlay device 100a,b can be individually positioned on the tendon 10 with respect to a given side of a laceration 15, and crimped to the tendon 10 such that each tendon overlay device's teeth 120 penetrate into lateral (i.e., non-dorsal) portions of the tendon 10. Such penetration of the teeth 120 into lateral portions of the tendon provides a first anchoring of each tendon overlay device 100 to the tendon 10. In various situations, each tendon overlay device 100 is typically positioned and crimped approximately 10 to 15 mm away from the laceration 15.

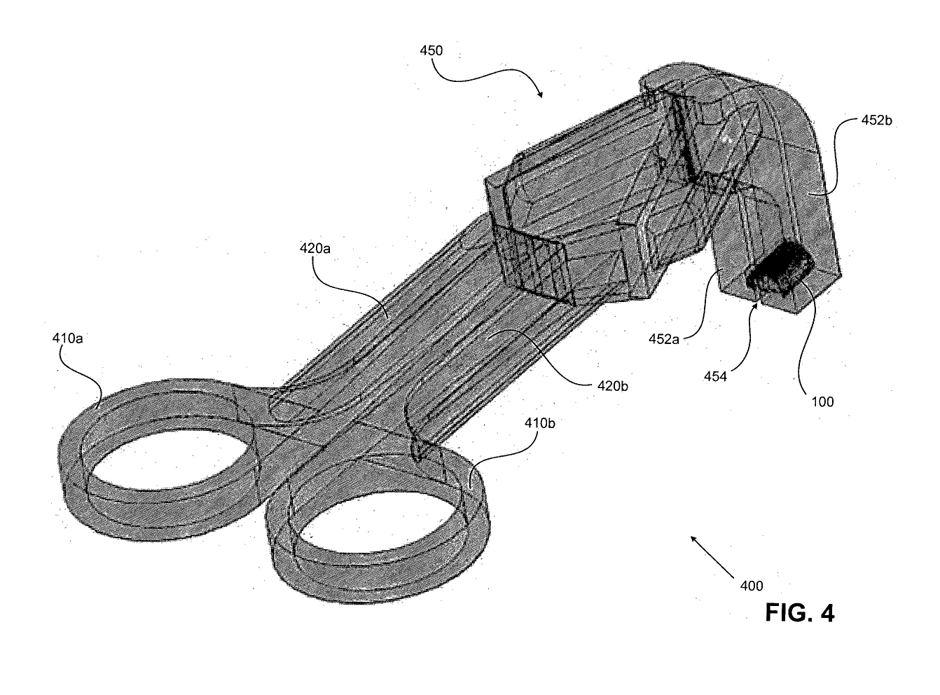

FIG. 4 is a schematic illustration of a representative tendon overlay device crimping tool 400 in accordance with an embodiment of the present disclosure. The crimping tool 400 is configured to (a) hold a tendon overlay device 100 while the tendon overlay device 100 partially surrounds an intended or target portion of a tendon 10; and (b) apply pressure or force to the tendon overlay device's outer surface 112 such that the tendon overlay device 100 is slightly mechanically deformed. As a result of such deformation, the tendon overlay device's first and second terminal edges 116a,b are simultaneously laterally displaced inwardly toward each other; and the teeth 120 carried by the first terminal edge 116a as well as the teeth 120 carried by the second terminal edge 116b are simultaneously laterally displaced inwardly toward each other.

As a result of this mechanical deformation, the teeth 120 of the tendon overlay device 100 can simultaneously penetrate into lateral portions of the tendon 10. Thus, the crimping of a given tendon overlay device 100 to the tendon 10 occurs by way of a single or one step crimping procedure.

In an embodiment, the crimping tool 400 includes a set of grippable or graspable handles 410a,b, which can include or be finger rings; a pair of elongate arm members 420a,b coupled to the handles 410a,b; and a head member 450 coupled to the arm members 420a,b. The head member 450 is configured to, hold or retain a tendon overlay device 100 between a first and a second closure arm 452a, 452b, which are separated by a closure gap 454 that defines a maximum crimping distance across which the closure arms 452a,b can be displaced toward each other, and hence a maximum lateral distance across which the tendon overlay device 100 can be laterally deformed during crimping to force or drive the teeth 120 into the tendon 10. In a representative implementation, this crimping distance can be approximately 0.5-5 mm (e.g., 1 mm-4 mm, or 1 mm, 2 mm, or 3 mm). In various embodiments, the crimping tool 400 has a structure that is analogous or generally analogous to that of a pair of forceps, in a manner understood by one of ordinary skill in the relevant art.

One or more crimping tools 400 can also form a portion of a tendon repair kit in accordance with an embodiment of the present disclosure. In some embodiments, a given crimping tool 400 can be designed to hold a tendon overlay device 100 of a specific outer diameter Dₒ, in which case any given crimping tool 400 corresponds to a tendon overlay devices 100 having a specific outer diameter Dₒ. Alternatively, different crimping tool head members 450 or closure arms 452a,b can be provided, each of which corresponds to a tendon overlay device 100 having a given outer diameter Dₒ, and each of which can be selectively coupled to the elongate arm members 420a,b and the handles 410a,b.

Once a first tendon overlay structure 100a has been crimped to a first tendon segment 10a on a first side of a tendon laceration 15, and a second tendon overlay structure 100b has been crimped to a second tendon segment 10b on a second side of the laceration 15, a set of sutures 200 can be routed into, through, and between particular suture openings 130a-d provided by each tendon overlay device 100. When tightened, such sutures 200 draw the first and second tendon overlay structures 100a,b as well as the first and second tendon segments 10a,b together to establish and maintain intimate contact between the first and second tendon segments 10a,b, and effectively form a set of mechanical or structural linkages between or spanning the first tendon overlay structure 100a, the second tendon overlay structure 100b, and portions of the tendon 10 itself. Epitendinous suture locks 210 can be provided to secure the overall tendon repair structure, which includes each tendon overlay device 100a,b and the sutures 200 disposed relative thereto) in place, in a manner understood by one having ordinary skill in the relevant art.

In general, 2-strand suture configurations are easier to perform compared to more complicated multi-strand suture techniques. With a suture technique such as that shown in FIGS. 1A-1B, the suture component passes deep to the longitudinal component of tendon fibers, so that the suture locks bundles of tendon fibers together when the suture is tensioned. Appropriate tensioning of the repair prevents gap formation at/across the repair site. The use of a tendon overlay/repair device in accordance with an embodiment of the present disclosure in combination with this or an analogous/similar type of suturing technique increases the tensile strength of the repair. Earlier studies were carried out to determine the tensile strength of the repair for 2-strand, 4-strand, and 8-strand suture techniques. Multi-strand suturing tensile strength was found to be stronger with increasing strand count; however, more complex multi-strand suturing results in increased technical difficulty, increased knot size, and increased friction along the tendon sheath. Thus, while multi-strand suturing techniques can be employed in association with a tendon overlay/repair device in accordance with the present disclosure, the suture technique shown in FIGS. 1A-1B works well or very well in the majority of situations.

Representative Biomedical Analysis

In a representative biomedical analysis, in vitro tendon repair was carried out on dissected and transected porcine tendons. Each tendon was repaired using a pair of tendon overlay devices 100a,b constructed in accordance with an embodiment of the present disclosure, and a commercially available suture (Teleflex 2-0). Additionally, an epitendonous suture was applied using 5-0 Prolene suture. Following repair, tendons were clamped to an Instron tensile strength testing system to determine ultimate tensile strength of the repaired tendons. Tendons repaired using tendon overlay devices 100 in accordance with embodiments of the present disclosure exhibited a maximum tensile strength of approximately 100 N.

Aspects of particular embodiments of the present disclosure address at least one aspect, problem, limitation, and/or disadvantage associated with exiting devices and techniques for tendon repair. While features, aspects, and/or advantages associated with certain embodiments have been described in the disclosure, other embodiments may also exhibit such features, aspects, and/or advantages, and not all embodiments need necessarily exhibit such features, aspects, and/or advantages to fall within the scope of the disclosure. It will be appreciated by a person of ordinary skill in the art that several of the above-disclosed systems, components, processes, or alternatives thereof, may be desirably combined into other different systems, components, processes, and/or applications. In addition, various modifications, alterations, and/or improvements may be made to various embodiments that are disclosed by a person of ordinary skill in the art within the scope of the present disclosure.

1. A tendon overlay device comprising:
an arcuate main body having an outer surface and an inner surface, each of the outer surface and the inner surface extending between a first terminal edge of the main body and a second terminal edge of the main body, wherein the inner surface of the main body excludes spikes and serrations that can penetrate into an outer surface of a tendon on which the inner surface of the main body is disposable;
a first plurality of teeth disposed along the first terminal edge of the main body; and
a second plurality of teeth disposed along the second terminal edge of the main body,
wherein each tooth within the first plurality of teeth projects downward away from the first terminal edge of the main body and is biased inward toward the second plurality of teeth, and
wherein each tooth within the second plurality of teeth projects downward away from the second terminal edge of the main body and is biased inward toward the first plurality of teeth.

2. The tendon overlay device of claim 1, wherein a central axis of the main body is definable which bisects the main body into symmetric arcuate halves, and wherein each tooth within each plurality of teeth is biased inward toward the central axis.

3. The tendon overlay device of claim 2, wherein each symmetric arcuate half is shaped in the form of a single arc.

4. The tendon overlay device of claim 2, wherein each tooth within each plurality of teeth is biased inward toward the central axis in accordance with a common angle.

5. The tendon overlay device of claim 1, wherein each of the outer surface of the main body and the inner surface of the main body form an arcuate segment of a circle.

6. The tendon overlay device of claim 5, wherein the circle has a predetermined diameter that is expected to substantially match the diameter of a tendon of a particular size such that the inner surface of the main body can matingly engage with a portion of a circumference of the tendon.

7. The tendon overlay device of claim 5, wherein the main body has a semi-cylindrical shape along a main body length L, the semi-cylindrical shape defined by a constant radius of curvature between the first terminal edge and the second terminal edge, and wherein each symmetric half of the main body exhibits a shape corresponding to a quarter cylinder.

8. The tendon overlay device of claim 1, wherein the main body has a thickness defined by a distance between the inner surface of the main body and the outer surface of the main body, and wherein the thickness is between approximately 0.20 mm and 0.30 mm.

9. The tendon overlay device of claim 2, wherein a ratio of a diameter corresponding to the arcuate main body and a distance to which each tooth within the first plurality of teeth and each tooth within the second plurality of teeth is configured to penetrate into a tendon is approximately 1.0 mm-1.3 mm.

10. The tendon overlay device of claim 1, wherein the main body is manufactured from a metal.

11. The tendon overlay device of claim 1, further comprising:
a first plurality of suture openings formed in the main body and disposed laterally away from the central axis of the main body proximate to the first terminal edge of the main body, above the first set of teeth; and
a second plurality of suture openings formed in the main body and disposed laterally away from the central axis of the main body proximate to the second terminal edge of the main body, above the second set of teeth.

12. The tendon overlay device of claim 11, wherein each of the first and second pluralities of suture openings include openings having at least one of different shapes and different cross sectional areas.

13. A tendon repair structure comprising:
a first tendon overlay device disposed on a first segment of a tendon corresponding to a first side of a laceration in the tendon;
a second tendon overlay device disposed on a second segment of the tendon corresponding to a second side of the laceration in the tendon; and
at least one sutures coupling the first tendon overlay device to the second tendon overlay device,
wherein each of the first and second tendon overlay devices comprises:
an arcuate main body having an outer surface and an inner surface, each of the outer surface and the inner surface extending between a first terminal edge of the main body and a second terminal edge of the main body, wherein the inner surface of the main body excludes spikes and serrations that can penetrate into an outer surface of a tendon on which the inner surface of the main body is disposable;
a first plurality of teeth disposed along the first terminal edge of the main body; and
a second plurality of teeth disposed along the second terminal edge of the main body,
wherein each tooth within the first plurality of teeth projects downward away from the first terminal edge of the main body and is biased inward toward the second plurality of teeth, and
wherein each tooth within the second plurality of teeth projects downward away from the second terminal edge of the main body and is biased inward toward the first plurality of teeth.

14. The tendon repair structure of claim 13, wherein each of the first and second tendon overlay devices further comprises a plurality of suture openings through which the at least one suture can be routed such that a force applied to the at least one suture draws the first and second tendon overlay devices toward each other and brings the first segment of the tendon into intimate contact with the second segment of the tendon.

15. A surgical kit for tendon repair, comprising:
at least one pair of tendon overlay devices, each tendon overlay device within the at least one pair of tendon overlay devices comprising:
an arcuate main body having an outer surface and an inner surface, each of the outer surface and the inner surface extending between a first terminal edge of the main body and a second terminal edge of the main body, wherein the inner surface of the main body excludes spikes and serrations that can penetrate into an outer surface of a tendon on which the inner surface of the main body is disposable;
a first plurality of teeth disposed along the first terminal edge of the main body; and
a second plurality of teeth disposed along the second terminal edge of the main body,
wherein each tooth within the first plurality of teeth projects downward away from the first terminal edge of the main body and is biased inward toward the second plurality of teeth, and
wherein each tooth within the second plurality of teeth projects downward away from the second terminal edge of the main body and is biased inward toward the first plurality of teeth,
wherein each pair of tendon overlay devices within the at least one pair of tendon overlay devices corresponds to a circle of a predetermined diameter that is expected to approximately correspond to a mammalian tendon.

16. The surgical kit of claim 15, wherein the circle of a predetermined diameter of each tendon overlay device comprises a circle that is expected to approximately correspond to a diameter of a human hand tendon within one of hand flexor tendon zone V, hand flexor tendon zones II to IV, and hand extensor tendon zones II to V.

17. The surgical kit of claim 15, further comprising at least one tendon sizing device having an arcuate measurement segment corresponding to a circle of a predetermined diameter that is expected to approximately correspond to a diameter of a mammalian tendon.

18. The surgical kit of claim 17, wherein the arcuate measurement segment of the at least one tendon sizing device corresponds to a circle that is expected to approximately correspond to a diameter of a human hand tendon within one of hand flexor tendon zone V, hand flexor tendon zones II to IV, and hand extensor tendon zones II to V.

19. The surgical kit of claim 15, further comprising a tendon overlay device crimping tool configured for (a) holding a tendon overlay device in position around a portion of a circumference of a tendon, and (b) applying a force to an outer surface of the tendon overlay device to thereby simultaneously drive the first plurality of teeth and the second plurality of teeth into lateral portions of the tendon.

20. The surgical kit of claim 19, wherein the tendon overlay device crimping tool comprises:
a set of handles graspable by a user;
a set of arm members coupled to the set of graspable handles;
a first closure arm and a second closure arm coupled to the set of arm members,
wherein the first and second closure arms are configured for holding the tendon overlay device in position on the tendon and applying the force to the outer surface of the tendon overlay device in response to user interaction with the set of handles, and
wherein the first and second closure arms are inwardly displaceable toward each other across a maximum crimping distance through which the closure arms can be displaced toward each other to laterally deform the tendon overlay device and simultaneously drive the first set of teeth and the second set of teeth into lateral portions of the tendon.
